(19) United States
(12) Patent Application Publication
(10) Pub. No.: US 2008/0027009 A1
(43) Pub. Date: Jan. 31, 2008

(54) Surfactant Preparation Having Reduced Enzyme Damaging

(75) Inventors: Andreas Schepky, Hamburg (DE); Albrecht Doerschner, Hamburg (DE); Ursula Holtzmann, Hamburg (DE); Katrin Counradi, Hamburg (DE); Stephan Ruppert, Hamburg (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 11/573,332

(22) PCT Filed: Mar. 24, 2005
(86) PCT No.: PCT/EP05/51392

§ 371 Date: May 14, 2007

(51) Int. Cl. A61K 31/131; A61K 31/20; A61K 31/7004; A61P 17/00

(52) U.S. Cl. 514/25; 514/558; 514/667; 514/673

(57) ABSTRACT

A method of protecting skin enzymes from the harmful effects of a cosmetic cleansing preparation which comprises water and one or more anionic surfactants. The method comprises using a cleansing preparation which has a pH value of from 4 to 7 and an SCTE (Stratum Comeum Tryptic Enzyme) value, determined following application of the preparation to human skin and standardized to a value of 100 for tap water, of from 65 to 95. The preparation comprises (i) one or more acyl-/dialkylethylenediamines which reduce the CMC of the one or more anionic surfactants and (ii) a buffer system of citric acid and citrate ions.

Cleansing the skin using surfactant-containing formulations should effectively remove surface lipids and dirt from the surface of the skin. The enzymes in the skin should be damaged as little as possible by this cleansing. The (anionic) surfactants and surfactant systems usually used deactivate the enzymes considerably. As a result, important metabolic physiological processes (desquamation etc.) of the skin are adversely affected.

For the purposes of the present specification, skin enzymes are enzymes which are present on the surface of the skin or close to the surface of the skin. Such enzymes may be: hydrolases, such as proteases, esterases, lipases, phosphatases, sulfatases and transglutaminases, but in particular proteases, such as the stratum corneum tryptic enzyme (SCTE). The most important stratum corneum enzymes known in the literature are indicated in tables 1 and 2 and below.

TABLE 1
Enzymes which degrade desmosomes and contribute to desquamation

| | Site | Reaction | |
|---|---|---|---|
| Enzyme | of activity | (barrier damage) | Literature |
| SCCE | SC (LB) | Cleavage of protein | Lundström, 1991 |
| | | bonds | Suzuki, 1994 |
| | | | Sondell, 1995 |
| | | | Chang-Yi, 1997 |
| Trypsin | SC | Cleavage of protein | Suzuki, 1994 |
| | | bonds ↑ | Chang-Yi, 1997 |
| Cathepsin | SG | Filaggrin degradation | Hara, 1993, |
| | | keratinization aid | Kawada, 1997 |
| Thiol protease | SC | | Yokozeki, 1987 |

TABLE 2
Enzymes which construct the barriers and
contribute to barrier homeostasis

| | Site of | Reaction | |
|---|---|---|---|
| Enzyme | activity | (barrier damage) | Literature |
| Phospholipase A₂ | SG-SC; | Release of fatty acids | Mauro, 1998 |
| | LB | and possibly | Mao-Qiang, |
| | | cholesterol from | 1995 Elias, |
| | | cholesterol esters | 1988 Menon, |
| | | | 1986 |
| Acidic lipase | SC, LB | Release of sterols | Menon, 1986 |
| | | | Elias, 1988 |
| Neutral lipase | SC, LB | Sterol - and fatty | Menon, 1986 |
| | | acid - release | |
| | | Regulation of protein | |
| | | kinases (differentiation) | |
| Sphingomyelinase | SC, LB | Provision of ceramides | Menon, 1986 |
| Ceramidase | SC | Provision of ceramides | Jin, 1994 |
| β-Gluocerebrosidase | SC | Conversion of | Holleran, 1992 |
| | | glycoceramides to | Mauro, 1998 |
| | | ceramides | |
| Steroid sulfatase | SC | Cholesterol release | Elias, 1988 |
| | | from cholesterol sulfate | |
| Sulfatases | SC | Precursor cleavage | Baden, 1980 |

Ammonia lyases play an important role during filaggrin degradation (Kuroda et al., 1979). So too do transglutaminases (Polakowska et al., 1991), which are essential for the formation of the “cornified envelope”. Phosphatases are the hydrolases with the highest overall activity in the stratum corneum.

Influence of enzymes on desquamation (see Schepky et al., 2004, Influence of cleansing on stratum corneum tryptic enzyme (SCTE) in human volunteers, Int. Journal of Cosmetic Science, 26, 245-253)

Rieger writes in 1994 in Cosmetic & Toiletries that the organization of the epidermis requires a chemical modification of constituents of the keratinocytes, inter alia in the lamellar bodies. Elias pointed to the need for hydrolytic (catabolic) enzymes in the skin. Proteases are required for the removal of desmosomal structures. If denaturing surfactants penetrate there and the enzyme activities are considerably impaired, a defective stratum corneum is the result. To maintain a constant thickness of the stratum corneum, the desquamation rate and the de novo production of the corneocytes must be balanced exactly. Egelrud demonstrated that the proteolysis by proteases is the central event in the desquamation process with the help of a plantar stratum corneum model. The enzymes best characterized with a function during desquamation are the stratum corneum chymotryptic enzyme (SCCE) and stratum corneum tryptic enzyme (SCTE). SCCE has a number of properties which correlate well with its role during desquamation in vivo: the pH profile of its catalytic unit, its specific inhibitor profile and its position in the tissue. SCTE has a similar role to SCCE during desquamation, but must additionally be able to activate inactive SCCE by hydrolysis. It is assumed that this enzyme cleaves autocatalytically from the inactive form to the active form. For both enzymes, it has been shown that topical application of specific inhibitors of these serine proteases (aprotinin and leupeptin) leads to more skin flakes in vivo. Sato et al. reported in 1998 that cholesterol-3 sulfate reduces both the activity of SCCE and also of SCTE through competitive inhibition. This is associated with reduced desquamation. Further proteases (cathepsin D) have been found in the stratum corneum, but are probably responsible primarily for the fine adjustment of desquamation.

Effects of Washing Products on the Skin Enzymes and Desquamation

Skin washing products comprise ionic surfactants, e.g. sodium dodecyl sulfate (SDS) or sodium lauryl ether sulfate (SLES). Such anionic surfactants are well known on account of their strong binding to globular proteins and into the skin as a result of electrostatic interaction of their charged group with the oppositely charged amino acid group of the proteins. Furthermore, the hydrophobic alkyl chain of the molecules of the surfactant also acts on the nonpolar zone of the globular proteins. As a consequence of this cooperative binding, surfactants induce conformational changes in the protein molecules which normally lead to the loss of biological, i.e. enzymatic, activity.

For SDS, this effect is even known as being irreversible. The interaction between denaturing surfactants and the enzymes important for skin desquamation can possibly lead to a defective SC.

This effect of washing products on the skin enzymes has already been investigated by quantifying the activity of the acidic phosphatase in skin strip biopsies of the human SC. Following treatment of the test subjects' skin with dilute solutions of various surfactants under realistic treatment conditions, the measured decrease in the activity of the acidic phosphatase in the SC indicated a significant correlation with the increasing dryness and flakiness of the skin.

For the purposes of the present specification, skin enzyme damaging means any form of inactivation of these enzymes by denaturation, inhibition or chemical degradation. If enzymes come into contact with surfactants, then it very often leads to denaturation. Prottey et al., in 1984, quantified the effect of surfactants on the acidic phosphatase of the stratum corneum (obtained by tape stripping) by measuring the phosphatase activity. Here, a reduction in enzyme activity as a result of denaturation of the enzyme was established. On the basis of further data, surfactant sensitivity is to be assumed.

Consequently, for the purposes of the present specification, enzyme protection is understood as meaning reduced damage/impairment of the described skin enzymes. The known products comprise, for example, mixtures of lauryl ether sulfate and alkylamidopropylbetaine.

Use of such products leads to partial denaturation of the skin enzymes and thus to skin damage since these enzymes have an important role in physiological terms.

The enzyme protection can be quantified as follows: firstly an ex vivo determination of the effect of surfactants on the trypsin activity in the human epidermis is carried out. Test subjects wash under supervision several times over 3 days using various products or water on different areas. 24 h later, the upper stratum corneum is extracted. The stratum corneum tryptic enzyme (SCTE) activity in the extract is measured. In parallel, the protein concentration of the extracts is determined in order to obtain the specific trypsin activity (correction for differing extraction of the areas).

DE 19838034 discloses mild shower products containing anionic surfactants and cocoamphoacetates. EP 1114639 A2 and EP 1114640 A2 disclose the use of certain cosurfactants in surface-active cleansing preparations for reducing the binding of certain surfactants to the surface of the skin. U.S. Pat. No. 6,468,515 B1 discloses hair care preparations. By contrast, nothing is disclosed about how skin enzyme protection against the disadvantageous effects of surface-active cleansing products can be achieved.

Surprisingly, it has now been found that the use of acyl-/dialkylethylenediamines, particularly preferably cocoamphoacetates, in cosmetic cleansing preparations which comprise water and anionic surfactants for skin enzyme protection, characterized in that the cleansing preparation has an SCTE value, standardized to tap water and measured after its application to the human skin, of from 65 to 95 and the acyl-/dialkylethylenediamine(s) reduce/reduces the CMC of the respective anionic surfactant, and a buffer system of citric acid and citrate ions is present and the pH of the preparation is adjusted to 4 to 7, overcomes the disadvantages of the prior art. Thus, the skin enzyme damage caused by anionic surfactants can be reduced, and effective enzyme protection is to be achieved.

The acyl-/dialkylethylenediamines are characterized in that they reduce the CMC of the respective anionic surfactant. As a result, the enzymes can better fulfill their essential tasks in the skin. Adjusting the product to a skin-neutral value with citric acid buffer can increase this effect.

It is preferred if the anionic surfactant or surfactants is/are chosen from the group of alkyl ether sulfates. It is preferred if the concentration of anionic surfactants is 5 to 15% by weight. It is preferred if the concentration of acyl-/dialkylethylenediamines is 0.5 to 8% by weight. The invention also covers a cosmetic cleansing preparation comprising 1 to 9% by weight of acyl-/dialkylethylenediamines, particularly preferably cocoamphoacetates, water and anionic surfactants, characterized by an SCTE value, standardized to tap water and measured following its application to the human skin, of from 65 to 95 where and the acyl-/dialkylethylenediamine(s) reduce/reduces the CMC of the respective anionic surfactant, and a buffer system of citric acid and citrate ions is present and the pH of the preparation is adjusted to 4 to 7. It is preferred if 0.8 to 1.2% by weight of PEG-40 hydrogenated castor oil are present. It is preferred if 0.3 to 0.5% by weight of PEG-200 hydrogenated glycerol palmitate are present. It is preferred if the ratio of anionic surfactant to acyl-/dialkylethylenediamines is 4/8 to 7.

Preparations according to the invention can comprise further surfactants. Particularly preferred surfactants are decyl glucoside, lauryl glucoside and lauryl citrate sulfosuccinate.

Cleansing preparations according to the invention are advantageously in the form of gels and comprise one or more gel formers and/or hydrocolloids. Particularly advantageous hydrocolloids are carbomers, xanthan gum, acylate copolymer, hydroxypropylcellulose and hydroxyethylcellulose.

The total amount of one or more hydrocolloids in the finished cosmetic or dermatological preparation is advantageously chosen to be less than 1.5% by weight, preferably between 0.1 and 1.0% by weight, based on the total weight of the preparations.

It is also advantageous to add complexing agents to the preparations according to the invention. The complexing agents are advantageously chosen from the group consisting of ethylenediaminetetraacetic acid (EDTA) and anions thereof, nitrilotriacetic acid (NTA) and anions thereof, hydroxyethylenediaminotriacetic acid (HOEDTA) and anions thereof, diethyleneaminopentaacetic acid (DPTA) and anions thereof, trans-1,2-diaminocyclohexanetetraacetic acid (CDTA) and anions thereof, tetrasodium iminodisuccinate, trisodium ethylenediaminedisuccinate.

Furthermore, conditioning auxiliaries may be present in the cosmetic cleansing compositions, e.g. in amounts of from 0.001 to 10% by weight, based on the total weight of the preparations.

Preferred conditioning auxiliaries include polyquaternium-10, polyquaternium-7 and quaternized guar gum.

Preservatives approved in food technology are to be used advantageously according to the invention.

The omission of a single constituent adversely affects the unique properties of the overall composition. For this reason, all of the stated constituents of the preparations according to the invention are obligatorily required in order to carry out the invention.

Having said all this, it is in some cases possible that the abovementioned concentration data are slightly exceeded or fallen short of and nevertheless preparations according to the invention are obtained. In view of the diversity of suitable components of such preparations, this comes as no surprise to the person skilled in the art, so that he is aware that such excesses or deficits do not depart from the essence of the present invention.

The examples below are intended to illustrate the present invention without limiting it. The numerical values in the examples are percentages by weight, based on the total weight of the respective preparations.

### EXAMPLES

1) Determination ex vivo of the Effect of Surfactants on the Trypsin Activity in the Human Epidermis

For standardizing the skin of the test subjects, the subjects were requested to use only a mild (with 3-10% myrystyl sulfate instead of lauryl ether sulfate) when washing for two weeks. After this preconditioning, the forearms were in each case divided into two test areas. The test areas were treated for three days in succession in each case 3 times daily with 1 ml of washing product (examples 1, 2, 3, 4, 5 or 6) or a tap water control for 45 s per area. After the treatment, the test area was rinsed off with tap water for 30 s and dried using a disposable paper towel. On the 1st and 2nd day, the areas were treated three times (morning, midday and afternoon), on the 3rd day they were treated twice (morning and midday). Up to three washing products were tested against one another and against water.

2) Extraction of the Skin Biopsy and Measurement of the SCTE Activity

On the 4th day, SC samples were stripped from the areas by means of a microscope slide coated with sugar solution. Later on, the corneocytes were detached from the microscope slide with PBS buffer and the specific SCTE activity was determined.

3) Stratum Corneum Tryptic Enzyme (SCTE) Activity Assay

100 μl of human skin extract were incubated for 24 h with 150 μl of N-t-BOC-Phe-Ser-Arg-7-amido-4-methylcoumarin (33 μM in PBS; Sigma, St Louis, USA) at 37° C. The SCTE-specific release of fluorescent 7-amino-4-methylcoumarin was ascertained using a fluorescence plate reader (filter ex=360 nm+40, em=460 nm+40 nm, CytoFluor 4000, PerSeptive Biosystems, Framingham, USA).

4) Measurement of the Protein Concentration

In order to calculate the specific trypsin activity of the extracts, the protein content was determined by means of the ninhydrin method following alkaline hydrolysis. The corneocyte solutions were evaporated to dryness and the proteins were hydrolyzed for 5 h at 150° C. with 2 ml of sodium hydroxide solution (6M). The solution was neutralized with 2 ml of hydrochloric acid (6M) and 1 ml of sodium propionic acid buffer (3.35 M, pH 5.5) was added. 50 μl of the lysate were then diluted with 450 μl of double-distilled water and incubated for 20 min at 70° C. with 25 μl of formic acid (0.4% (v/v)) and 500 μl of ninhydrin solution (2% (w/v) ninhydrin in 3.35 M sodium propionic acid buffer with 50% (v/v) ethylene glycol monomethyl ether (Sigma, St Louis, USA)). After cooling, 5 ml of ethanol (50% (v/v) in double-distilled water) were added. The absorption was measured at a wavelength of 570 nm using a spectrophotometer (UVICON 942, Kontron, Milan, Italy) and the corresponding protein concentration was calculated.

5) Shower Gels

| | 1 | 2 | 3 | 4 | 5 | 6 | Control |
|---|---|---|---|---|---|---|---|
| Sodium laureth sulfate | — | 2% | — | 8% | 10% | 12% | 10% |
| Sodium myreth sulfate | 5% | 4% | 6% | — | — | — | — |
| Lauryl glucoside | 2.5% | — | — | — | — | 1.5% | — |
| Decyl glucoside | 2.0% | — | — | 0.5% | — | — | — |
| Sodium cocoamphoacetate | 6.5% | 7% | 8% | 6% | 4% | 2% | — |
| PEG-200 hydrogenated | 0.4% | 0.4% | 0.4% | 0.4% | 0.4% | 0.4% | 0.4% |
| glyceryl palmitate | | | | | | | |
| PEG-40 hydrogenated castor | 1% | 1% | 1% | 1% | 1% | 1% | 1% |
| oil | | | | | | | |
| Diammonium citrate | 0.12% | 0.12% | 0.12% | — | 0.12% | — | 0.12% |
| Polyquaternium-10 | 0.3% | — | — | 0.1% | 0.2% | — | 0.2% |
| Sodium benzoate | 0.3% | 0.3% | 0.3% | 0.3% | 0.3% | 0.3% | 0.3% |
| Sodium salicylate | 0.2% | 0.2% | 0.2% | 0.2% | 0.2% | 0.2% | 0.2% |
| Citric acid | 1.2% | 1.2% | 1.2% | q.s. | 1.2% | q.s. | 1.2% |
| Perfume | q.s. | q.s. | q.s. | q.s. | q.s. | — | q.s. |
| Water | ad 100 | ad 100 | ad 100 | ad 100 | ad 100 | ad 100 | ad 100 |
| pH | 4.8 | 5.0 | 5.0 | 5.6 | 5.3 | 6.5 | 5.3 |
| SCTE value standardized to | | 73 | 82 | | 70 | | 55 |
| tap water = 100 | | | | | | | |

1. The use of acyl-/dialkylethylenediamines, particularly preferably cocoamphoacetates in cosmetic cleansing preparations which comprise water and anionic surfactants for skin enzyme protection, characterized in that the cleansing preparation has an SCTE value, which is standardized to tap water and measured after its application to the human skin, of from 65 to 95 and the acyl-/dialkylethylenediamine(s) reduce/reduces the CMC of the respective anionic surfactant, and a buffer system of citric acid and citrate ions is present and the pH of the preparation is adjusted to 4 to 7.

2. The use as claimed in one of the preceding claims, characterized in that the anionic surfactant(s) is/are chosen from the group of alkyl ether sulfates.

3. The use as claimed in one of the preceding claims, characterized in that the concentration of anionic surfactants is 5 to 15% by weight.

4. The use as claimed in one of the preceding claims, characterized in that the concentration of acyl-/dialkylethylenediamines A is 0.5 to 8% by weight.

5. A cosmetic cleansing preparation comprising 1 to 9% by weight of acyl-/dialkyl-ethylenediamines, particularly preferably cocoamphoacetates, water and anionic surfactants, characterized by an SCTE value, standardized to tap water and measured following its application to the human skin, of from 65 to 95 where and the acyl-/dialkylethylenediamine(s) reduce/reduces the CMC of the respective anionic surfactant, and a buffer system of citric acid and citrate ions is present and the pH of the preparation is adjusted to 4 to 7.

6. The preparation as claimed in claim 5, characterized in that 0.8 to 1.2% by weight of PEG-40 hydrogenated castor oil are present.

7. The preparation as claimed in claim 5 to 6, characterized in that 0.3 to 0.5% by weight of PEG-200 hydrogenated glyceryl palmitate are present.

8. The preparation or the use as claimed in one of the preceding claims, characterized in that the ratio of anionic surfactant to acyl-/dialkylethylenediamines is 4/8 to 7.

1-8. (canceled)

9. A method of protecting skin enzymes from the harmful effects of a cosmetic cleansing preparation which comprises water and one or more anionic surfactants, wherein the method comprises using a cleansing preparation which has a pH value of from 4 to 7 and an SCTE (Stratum Comeum Tryptic Enzyme) value, determined following application of the preparation to human skin and standardized to a value of 100 for tap water, of from 65 to 95, the preparation comprising (i) one or more acyl-/dialkylethylenediamines which reduce a CMC of the one or more anionic surfactants and (ii) a buffer system of citric acid and citrate ions.

10. The method of claim 9, wherein the one or more acyl-/dialkylethylenediamines comprise one or more cocoamphoacetates.

11. The method of claim 9, wherein the one or more anionic surfactants comprise one or more alkyl ether sulfates.

12. The method of claim 9, wherein the preparation comprises from 5% to 15% by weight of the one or more anionic surfactants.

13. The method of claim 9, wherein the preparation comprises from 0.5% to 8% by weight of the one or more acyl-/dialkylethylenediamines.

14. A cosmetic cleansing preparation, wherein the preparation comprises water, one or more anionic surfactants, from 1% to 9% by weight of one or more acyl-/dialkylethylenediamines which reduce a CMC of the one or more anionic surfactants, and a buffer system of citric acid and citrate anions and wherein the preparation has a pH value of from 4 to 7 and an SCTE (Stratum Comeum Tryptic Enzyme) value, determined following application of the preparation to human skin and standardized to a value of 100 for tap water, of from 65 to 95.

15. The preparation of claim 14, wherein the one or more acyl-/dialkylethylenediamines comprise one or more cocoamphoacetates.

16. The preparation of claim 14, wherein the one or more anionic surfactants comprise one or more alkyl ether sulfates.

17. The preparation of claim 14, wherein the preparation comprises from 5% to 15% by weight of the one or more anionic surfactants.

18. The preparation of claim 14, wherein a ratio of the one or more anionic surfactants to the one or more acyl-/dialkylethylenediamines is from 4/8 to 7.

19. The preparation of claim 14, wherein the preparation further comprises at least one of from 0.8% to 1.2% by weight of PEG-40 hydrogenated castor oil and from 0.3% to 0.5% by weight of PEG-200 hydrogenated glyceryl palmitate.

20. The preparation of claim 14, wherein the preparation further comprises at least one of decyl glucoside, lauryl glucoside and lauryl citrate sulfosuccinate.

21. The preparation of claim 14, wherein the preparation further comprises one or more hydrocolloids.

22. The preparation of claim 14, wherein the preparation further comprises one or more complexing agents.

23. A cosmetic cleansing preparation, wherein the preparation comprises water, from 5% to 15% by weight of one or more anionic surfactants which comprise one or more alkyl ether sulfates, from 1% to 8% by weight of one or more cocoamphoacetates which reduce a CMC of the one or more anionic surfactants, and a buffer system of citric acid and citrate anions and wherein the preparation has a pH value of from 4 to 7 and an SCTE (Stratum Comeum Tryptic Enzyme) value, determined following application of the preparation to human skin and standardized to a value of 100 for tap water, of from 65 to 95.

24. The preparation of claim 23, wherein a ratio of the one or more anionic surfactants to the one or more cocoamphoacetates is from 4/8 to 7.

25. The preparation of claim 23, wherein the preparation further comprises at least one of from 0.8% to 1.2% by weight of PEG-40 hydrogenated castor oil and from 0.3% to 0.5% by weight of PEG-200 hydrogenated glyceryl palmitate.

26. The preparation of claim 23, wherein the preparation further comprises at least one of decyl glucoside, lauryl glucoside and lauryl citrate sulfosuccinate.

27. The preparation of claim 23, wherein the preparation further comprises from 0.1% to 1.5% by weight of one or more hydrocolloids.

28. The preparation of claim 23, wherein the preparation further comprises one or more complexing agents.
